(19) United States
(12) Patent Application Publication
(10) Pub. No.: US 2025/0221664 A1
(43) Pub. Date: Jul. 10, 2025

(54) EMOTIONAL IMPACT ANALYSIS SYSTEM BASED ON CARBON DIOXIDE EXPOSURE

(71) Applicant: Tianjin University, Tianjin (CN)

(72) Inventors: Liqun CHEN, Tianjin (CN); Yukai YANG, Tianjin (CN); Jiaxing ZHANG, Tianjin (CN); Dong MING, Tianjin (CN); Yue CHEN, Tianjin (CN)

(21) Appl. No.: 19/011,797

(22) Filed: Jan. 7, 2025

(30) Foreign Application Priority Data
Jan. 10, 2024 (CN) 202410036937.8

(51) Int. Cl. A61B 5/16; A61B 5/00; A61B 5/0205; A61B 5/021; A61B 5/024; A61B 5/145; A61B 5/378; A61B 5/398; A61K 33/00; A61P 25/28

(52) U.S. Cl. CPC A61B 5/4884; A61B 5/165; A61B 5/0205; A61B 5/378

(57) ABSTRACT

An emotional impact analysis system based on carbon dioxide exposure is provided, including a closed experimental space, a high-pressure gas cylinder filled with pure carbon dioxide and a carbon dioxide analyzer, an emotional stimulation and feedback device, a biosensor acquisition device and an analysis device; the analysis device is connected with the carbon dioxide analyzer, the emotional stimulation and feedback device and the biosensor acquisition device, and is arranged outside the closed experimental space.

### CROSS-REFERENCE TO RELATED APPLICATIONS

This application claims priority to Chinese Patent Application No. 202410036937.8, filed on Jan. 10, 2024, the contents of which are hereby incorporated by reference.

### TECHNICAL FIELD

The disclosure relates to an emotional impact analysis system based on carbon dioxide exposure.

### BACKGROUND

Emotion has always been the focus of psychological research. It is difficult to describe emotions directly, but may be detected in many ways, such as expressing behaviors, physiological indicators and self-assessment. Many theorists and physiologists try to define emotions in a practical way. In 1980, Russell put forward the valence-arousal model, in which he distributed 16 kinds of emotions on a two-dimensional plane, namely arousal (from pleasant state to unpleasant state) and valence (from calm state to excited state). The emotional stimulus database developed according to this model has been widely used in psychology, psychophysiology and neuroscience research.

The following physiological indexes and emotions are all interactive: (1) electroencephalogram: firstly, emotional reactions mainly occur in frontal and temporal lobes; secondly, the wave bands related to emotional regulation are mainly theta wave, beta wave and gamma wave, which may be used to explore whether the interaction between these three waves (positive or negative picture stimulation) is significant. (2) Cortisol: High cortisol level may aggravate negative emotions and even lead to increased anxiety or pain; moreover, some studies show that cortisol may also play a role in regulating positive emotions and social connections. Therefore, high cortisol indicates negative emotions, while low cortisol indicates positive emotions. (3) Blood pressure: Long-term high-load stress, mental tension, anxiety, depression, sadness, anger, excitement and other emotions will excite sympathetic nerves, and sudden changes in emotions, such as excitement, panic, anxiety and mental tension, may increase blood pressure; satisfaction, peace of mind and happiness may lower blood pressure. Appreciating movies, TV plays and music with cheerful plots and clear lyrics may calm the nerves and lower blood pressure; Watching intense, thrilling and scary movies and football games will induce a sharp rise in blood pressure.

Carbon dioxide is a common greenhouse gas, and the concentration of carbon dioxide in indoor environment will be higher, especially in vehicles such as cars and engine rooms. For example, when the concentration of carbon dioxide in the body rises, the body will have an early warning of suffocation, resulting in tension, anxiety and irritability. Other studies have shown that high concentrations of carbon dioxide may activate the amygdala in the brain, which controls emotional reactions such as fear and panic.

However, the prior art has not disclosed how the carbon dioxide concentration affects people's emotions and how the human body submits emotions after the influence.

### SUMMARY

The disclosure aims to overcome the shortcomings of the prior art and provide an emotional impact analysis system based on carbon dioxide exposure.

The objective of the disclosure is achieved through the following technical scheme.

In a first aspect of the present disclosure, there is provided an emotional impact analysis system based on carbon dioxide exposure, including:
a closed experimental space;
a high-pressure gas cylinder filled with pure carbon dioxide and a carbon dioxide analyzer, where a gas outlet of the high-pressure gas cylinder and the carbon dioxide analyzer are both arranged in the closed experimental space for providing multiple preset carbon dioxide environments with different carbon dioxide concentrations in the closed experimental space;
an emotional stimulation and feedback device arranged in the closed experimental space and used for providing emotional stimulation pictures with established specifications to subjects and receiving emotional feedback results of the subjects after emotional stimulation;
a biosensor acquisition device including a blood pressure-heart rate acquisition device, a cortisol acquisition device and an electroencephalogram acquisition device, arranged in the closed experimental space and used for acquiring biological signals used by the subjects to express emotions;
an analysis device connected with the carbon dioxide analyzer, the emotional stimulation and feedback device and the biosensor acquisition device and arranged outside the closed experimental space, used to monitor the carbon dioxide environments in the closed experimental space, control a normal work of the emotional stimulation and feedback device and the biosensor acquisition device, analyze emotional influence and emotional adjustment of the subjects in different carbon dioxide environments according to data of the emotional stimulation and feedback device and the biosensor acquisition device.

Further, the preset carbon dioxide environments include a carbon dioxide environment with a low level of carbon dioxide concentration and a carbon dioxide environment with a high level of carbon dioxide concentration, the low level of carbon dioxide concentration is close to a carbon dioxide concentration in a normal ventilation room, and the high level of carbon dioxide concentration is a limit value of a maximum occupational exposure in an 8-hour working day.

Further, the emotional stimulation pictures of the established specifications include negative pictures and positive pictures with a same number, and all emotional stimulation pictures are randomly presented.

Further, emotional feedback results of the subjects after the emotional stimulation include a judgment feedback result of the subjects to judge whether presented picture makes them feel happy or not, and a feedback time to judge a feedback result.

Further, the blood pressure-heart rate acquisition device and the cortisol acquisition device acquire before and after the emotional stimulation and feedback device is used; the electroencephalogram acquisition device acquires the electroencephalogram during use of the emotional stimulation and feedback device, that is, the emotional stimulation and feedback device receives the emotional feedback results of the subjects after the emotional stimulation and the electroencephalogram acquisition device acquires electroencephalogram signals at a same time.

Further, when the analysis device analyzes the electroencephalogram acquisition device, including following steps:
segmenting the electroencephalogram signals collected by the electroencephalogram acquisition device, corresponding to segments corresponding to tasks of each of the emotional stimulation pictures;
performing baseline correction for each of the segments, including subtracting an average amplitude of a baseline within a specific time window; and
processing signals after the baseline correction by Fourier transform or continuous wavelet transform, and analyzing spectrum.

Further, before segmenting the electroencephalogram signals collected by the electroencephalogram acquisition device, further including data preprocessing, including following steps:
removing unnecessary electrode data in the electroencephalogram signals, including vertical electrooculogram VEOG and horizontal electrooculogram HEOG;
adopting Butterworth filter of 0.5-40 Hz to carry out band-pass filtering on the electroencephalogram signals; and
using independent component analysis (ICA) to remove artifacts caused by eyeball movement and blinking.

Further, a duration corresponding to the segments is −1 second to 2 seconds, and the specific time window is −1 second to 0 second.

Furthermore, an outlier analysis step is further included between the segments and the baseline correction, that is, outliers with positive amplitude or negative amplitude exceeding 100 are deleted.

Furthermore, for a same subject, experiments in different preset carbon dioxide environments need to be separated for a period of time.

The disclosure has the following beneficial effects.

In an exemplary embodiment of the present disclosure, a closed experimental space is provided so that the space may adjust the carbon dioxide environment in the emotional experiment process, emotional stimulation and biological signal collection are carried out on the subjects by using the emotional stimulation and feedback device and the biosensor acquisition device, and finally the emotional influence and emotional adjustment of the subjects in different carbon dioxide environments are analyzed by the analysis device, so that it is found that carbon dioxide has an influence on the emotional processing ability. And the specific results of the analysis show that this influence is not only negative, but also has some positive aspects to consider.

### BRIEF DESCRIPTION OF THE DRAWINGS

FIG. 1 is a schematic structural diagram of an emotional impact analysis system based on carbon dioxide exposure provided in an exemplary embodiment of the present disclosure.

FIG. 2 is a process diagram of an emotional task provided in an exemplary embodiment of the present disclosure.

### DETAILED DESCRIPTION OF THE EMBODIMENTS

The technical scheme of the present disclosure will be described clearly and completely with the attached drawings. Obviously, the described embodiment is a part of the embodiment of the present disclosure, but not the whole embodiment. Based on the embodiments in the present disclosure, all other embodiments obtained by people of ordinary skill in the field without creative labor belong to the protection scope of the present disclosure.

In the description of the present disclosure, it should be noted that the directions or positional relationships indicated by “center”, “upper”, “lower”, “left”, “right”, “vertical”, “horizontal”, “inner” and “outer” are based on the directions or positional relationships described in the attached drawings, which are only for the convenience of describing the present disclosure and simplifying the description, and are not indicated or implied. In addition, belonging to “first” and “second” are only used for descriptive objectives and cannot be understood as indicating or implying relative importance.

In the description of the present disclosure, it should be noted that unless otherwise specified and limited, “installation”, “connection” and “connection” should be broadly understood, for example, they may be fixed connection, detachable connection or integrated connection; it may be mechanically connected or electrically connected; it may be directly connected, may also be indirectly connected through an intermediate medium, and may be connected inside two elements. For those skilled in the art, the specific meanings of the above terms in the present disclosure may be understood in specific situations.

In addition, the technical features involved in different embodiments of the present disclosure described below may be combined with each other as long as they do not conflict with each other.

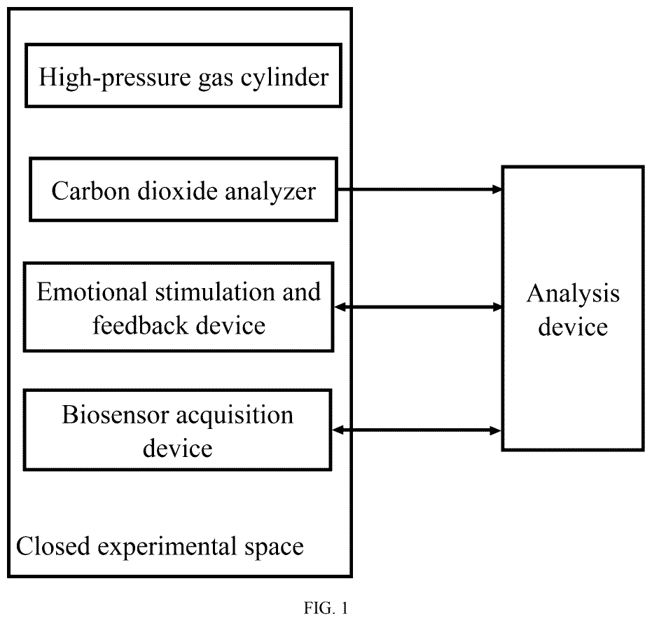

FIG. 1 provides a schematic structural diagram of an emotional impact analysis system based on carbon dioxide exposure in an exemplary embodiment of the present disclosure, including:
a closed experimental space;
a high-pressure gas cylinder filled with pure carbon dioxide and a carbon dioxide analyzer, where a gas outlet of the high-pressure gas cylinder and the carbon dioxide analyzer are both arranged in the closed experimental space for providing multiple preset carbon dioxide environments with different carbon dioxide concentrations in the closed experimental space;
an emotional stimulation and feedback device arranged in the closed experimental space and used for providing emotional stimulation pictures with established specifications to subjects and receiving emotional feedback results of the subjects after emotional stimulation;
a biosensor acquisition device including a blood pressure-heart rate acquisition device, a cortisol acquisition device and an electroencephalogram acquisition device, arranged in the closed experimental space and used for acquiring biological signals used by the subjects to express emotions;
an analysis device connected with the carbon dioxide analyzer, the emotional stimulation and feedback device and the biosensor acquisition device and arranged outside the closed experimental space, used to monitor the carbon dioxide environments in the closed experimental space, control a normal work of the emotional stimulation and feedback device and the biosensor acquisition device, analyze emotional influence and emotional adjustment of the subjects in different carbon dioxide environments according to data of the emotional stimulation and feedback device and the biosensor acquisition device.

Specifically, in this exemplary embodiment, a closed experimental space is provided so that the space may adjust the carbon dioxide environment in the emotional experiment process, emotional stimulation and biological signal collection are carried out on the subjects by using the emotional stimulation and feedback device and the biosensor acquisition device, and finally the emotional influence and emotional adjustment of the subjects in different carbon dioxide environments are analyzed by the analysis device, so that it is found that carbon dioxide has an influence on the emotional processing ability. And the specific results of the analysis show that this influence is not only negative, but also has some positive aspects to consider.

The following contents will elaborate the experimental process in detail.

First, the experimental subjects:
participants are recruited in a student group through social media. Exclusion criteria include self-report or suspicion 1) history of chronic diseases such as hypertension, diabetes, cardiovascular and cerebrovascular diseases; 2) other diseases, such as brain injury, or nervous system diseases that may affect brain function and structure; 3) the history of claustrophobia; 4) smoking history and respiratory history.

Specifically, in this exemplary embodiment, 30 healthy college students (15 males and 15 females), aged 18-28 and right-handed, are recruited.

Second, the physiological data collection of the subjects before the experiment

Before inviting the subjects into the closed experimental space, the required carbon dioxide concentration in the closed experimental space is finely adjusted and controlled. After the subjects are invited to enter the closed experimental space, the body temperature is measured by mercury thermometer, the blood pressure and heart rate are measured by OMRON electronic sphygmomanometer, and saliva samples are collected as baseline data. Among them, the subjects will rest for 3 minutes before measuring blood pressure and heart rate to avoid the influence caused by movement before entering the closed experimental space, and the subjects will wear EEG measuring equipment. Subjects should adapt to the closed experimental space environment for 90 minutes before entering the closed experimental space and starting the emotional experience task.

Third, environmental exposure

The carbon dioxide exposure experiment is conducted in a closed experimental space (3.2 m×3.2 m×2.5 m). Pure carbon dioxide is injected into a high-pressure gas cylinder to adjust the carbon dioxide concentration, and the temperature and carbon dioxide concentration are detected in real time by three Telaire TEL-7001 carbon dioxide analyzers, which are placed in front of the right side of the subject and behind the two sides of the subject. During the exposure, the temperature is controlled to some extent.

In this exemplary embodiment, two levels of carbon dioxide concentration are set to analyze the influence on people's emotional experience with the change of carbon dioxide concentration. Among them, the low-level carbon dioxide concentration is preferably 600 ppm, which is similar to the carbon dioxide concentration in the normal ventilation room; the high-level carbon dioxide concentration is preferably 5000 ppm, which is the maximum occupational exposure limit recommended by the Occupational Safety and Health Administration and the American Government Conference of Industrial Hygienists for an 8-hour working day.

When exposed to a high level of carbon dioxide concentration, a high-pressure gas cylinder is used to inject carbon dioxide gas with a purity of 99% into the closed experimental space, and the subjects are invited to enter when the carbon dioxide concentration is about stable at the set value. Two experimenters and one subject stay in the closed experimental space at the same time. During this period, the concentration of carbon dioxide will be monitored at any time and the attenuated carbon dioxide will be compensated.

Fourth, emotional tasks

In this exemplary embodiment, when in an emotional task, subjects are asked to watch 66 emotional stimulation pictures with established specifications, and 33 negative pictures (valence range 1.2-2.91, Mean=2.1426, SD=0.48816, arousal range 4.01-5.32, Mean=4.6329, SD=0.33013) and 33 positive pictures (valence range 5.6-6.49, mean=6.0039, SD=0.2364, arousal range 4.01-5.03, mean=4.3814, SD=0.29949) are selected.

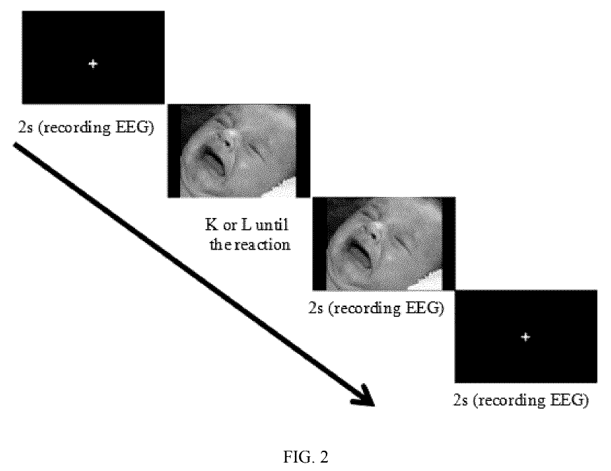

E-prime is used to create an experimental program, and all emotional stimulation pictures are presented randomly. The experimental task requires the subjects to judge whether the pictures presented by the emotional stimulation and feedback device make the subjects feel happy, the subject will press the “K” key if they feel happy, and press the “L” key if they don't feel happy. The reaction time and accuracy of the subjects were recorded, as shown in FIG. 2.

When the subject completes the emotional task, the electroencephalogram acquisition device will record the electroencephalogram of the subjects. After completing the emotional task, the experimenter will measure the exposed body temperature, blood pressure, heart rate and collect saliva samples from the subjects.

Optionally, in an exemplary embodiment, the two levels of carbon dioxide concentration are exposed one week apart, and the exposure sequence is balanced.

The electroencephalogram signal was recorded by grael portable amplifier, which has 32 channels (Fp1, Fp2, F7, F3, Fz, F4, F8, FT7, FC3, FCz, FC4, FT8, M1, T7, C3, Cz, C4, T8, M2, TP7, CP3, CPz, CP4, TP8, P7, P3, PZ, P4, P8, O1, O2) located according to the international 10-20 system. The sampling frequency of electroencephalogram is 512 Hz, and the impedance of all channels is lower than 20 kΩ.

Five, data processing

When analyzing the electroencephalogram acquisition device, the analysis device includes the following steps:
segmenting the electroencephalogram signals collected by the electroencephalogram acquisition device, corresponding to segments corresponding to tasks of each of the emotional stimulation pictures;
performing baseline correction for each of the segments, including subtracting an average amplitude of a baseline within a specific time window; where by subtracting this baseline from the converted data, the relative change of power spectrum with time may be effectively isolating and analyzing; and
processing signals after the baseline correction by Fourier transform or continuous wavelet transform, and analyzing spectrum. This transformation allows to calculate the power spectrum of each frequency band of interest, including delta (0.5-4 Hz), theta (4-8 Hz), alpha (8-13 Hz), beta (14-27 Hz) and gamma (27-40 Hz). By detecting the power in these specific frequency ranges, the activity and regulation of different nerve oscillations in the experimental task may be observed.

Optionally, in an exemplary embodiment, before segmenting the electroencephalogram signals collected by the electroencephalogram acquisition device, data preprocessing is further included, including the following steps:
removing unnecessary electrode data in the electroencephalogram signals, including vertical electrooculogram VEOG and horizontal electrooculogram HEOG;
adopting Butterworth filter of 0.5-40 Hz to carry out band-pass filtering on the electroencephalogram signals; and
using independent component analysis (ICA) to remove artifacts caused by eyeball movement and blinking. During this substep, each participant identifies and removes about 1-2 independent components. This is helpful to eliminate the confusion effect caused by eye-related activities in electroencephalogram recording.

Optionally, in an exemplary embodiment, a duration corresponding to the segments is −1 second to 2 seconds, and the specific time window is −1 second to 0 second.

Optionally, in an exemplary embodiment, an outlier analysis step is further included between the segments and the baseline correction, that is, outliers with positive amplitude or negative amplitude exceeding 100 are deleted to ensure data integrity and minimize the influence of extreme values.

Six, experimental phenomena

1. Blood pressure and heart rate

This exemplary embodiment found that the results of blood pressure, heart rate and saliva cortisol level are different from those in the prior art. The prior art reports that when a subject is exposed to high carbon dioxide concentration, blood pressure and heart rate will increase significantly. In our research, it is found that the blood pressure decreased when the subjects are exposed to high carbon dioxide concentration.

It is well known that blood pressure is closely related to emotions. When people are emotional, blood pressure will increase, and when people are emotionally stable, blood pressure will also decrease. Therefore, according to the results of this exemplary embodiment, it is inferred that exposure to a certain concentration of carbon dioxide for a certain period of time will help people reduce the negative impact of blood pressure caused by emotions, thus reducing the risk of cardiovascular diseases.

2. Cortisol

Another important finding of this exemplary embodiment is the significant increase of salivary phlogistic alcohol level when the subject is exposed to high carbon dioxide concentration.

On the one hand, the physiological secretion of cortisol occurs in a regular 24-hour (circadian rhythm) pattern, which is higher in the morning and decreases with the progress of the day. It can be seen that the emotional task in this exemplary embodiment causes changes in cortisol secretion. Cortisol is a major stress biomarker controlled by hypothalamic pituitary adrenal (HPA) axis. High cortisol levels caused by carbon dioxide exposure may aggravate negative emotions and even lead to increased anxiety or pain.

On the other hand, some studies show that cortisol may also play a role in regulating positive emotions and social connections. Therefore, it is very important to study how the change of cortisol level caused by carbon dioxide affects different aspects of emotional experience. In addition, it is also meaningful to explore whether there are individual differences in people's responses to changes in cortisol levels caused by carbon dioxide. Factors such as age, gender and existing emotional state may affect the influence of carbon dioxide on emotions.

In a word, more research is needed to fully understand the complex interaction among carbon dioxide exposure, cortisol level and emotional response. This knowledge is not only helpful to understand the influence of environmental factors on emotions, but also meaningful to create a healthier and more conducive environment for emotional health.

3. EEG

Beta and gamma waves in electroencephalogram are important bands for processing and processing emotions. Beta wave is related to emotional regulation and emotional stability, and people with emotional stability usually produce more beta wave activity in the cerebral cortex, which may mean that beta wave plays a role in emotional regulation and emotional processing. In addition, Gamma wave plays an important role in the process of perception and consciousness. When people perceive, pay attention to and realize external stimuli, the activity level of Gamma waves usually increases, which may be related to the rapid processing and integration of stimuli by the brain.

In this exemplary embodiment, however, it is found that the increase of carbon dioxide concentration will make the beta and gamma waves more active. This shows that high concentration of carbon dioxide exposure may affect the brain's ability to regulate emotions and effectively integrate stimuli.

To sum up, it is found in this exemplary embodiment that carbon dioxide has an impact on the processing ability of emotional processing, and this impact is not only negative, but also has some positive aspects to consider. According to the research results, although the increase of carbon dioxide level may lead to prolonged emotional processing time and increased cognitive efforts, it seems to have a positive impact on emotional stability.

Obviously, the above-mentioned embodiments are only an example for clear explanation, not a limitation of the implementation. For ordinary technicians in the field, other different forms of changes or variations may be made on the basis of the above description. It is not necessary and impossible to list all the embodiments here. However, the obvious changes or variations derived therefrom are still within the scope of protection defined by claims of the disclosure.

What is claimed is:

1. An emotional impact analysis system based on carbon dioxide exposure, comprising:
a closed experimental space;
a high-pressure gas cylinder filled with pure carbon dioxide and a carbon dioxide analyzer, wherein a gas outlet of the high-pressure gas cylinder and the carbon dioxide analyzer are both arranged in the closed experimental space for providing a plurality of preset carbon dioxide environments with different carbon dioxide concentrations in the closed experimental space;
an emotional stimulation and feedback device arranged in the closed experimental space and used for providing emotional stimulation pictures with established specifications to subjects and receiving emotional feedback results of the subjects after emotional stimulation; wherein the emotional stimulation pictures of the established specifications comprise negative pictures and positive pictures with a same number, and all the emotional stimulation pictures are randomly presented; the emotional feedback results of the subjects after the emotional stimulation comprise a judgment feedback result of the subjects to judge whether a presented picture makes the subjects feel happy or not, and a feedback time to judge a feedback result;
a biosensor acquisition device comprising a blood pressure-heart rate acquisition device, a cortisol acquisition device and an electroencephalogram acquisition device, arranged in the closed experimental space and used for acquiring biological signals used by the subjects to express emotions; wherein the blood pressure-heart rate acquisition device and the cortisol acquisition device acquire before and after the emotional stimulation and feedback device is used; the electroencephalogram acquisition device performs acquisition during use of the emotional stimulation and feedback device, that is, the emotional stimulation and feedback device receives the emotional feedback results of the subjects after the emotional stimulation and the electroencephalogram acquisition device acquires electroencephalogram signals at a same time; and
an analysis device connected with the carbon dioxide analyzer, the emotional stimulation and feedback device and the biosensor acquisition device and arranged outside the closed experimental space, used to monitor the carbon dioxide environments in the closed experimental space, control a normal work of the emotional stimulation and feedback device and the biosensor acquisition device, analyze emotional influence and emotional adjustment of the subjects in different carbon dioxide environments according to data of the emotional stimulation and feedback device and the biosensor acquisition device, and explore influence of carbon dioxide on an emotional processing ability.

2. The emotional impact analysis system based on carbon dioxide exposure according to claim 1, wherein the preset carbon dioxide environments comprise a carbon dioxide environment with a low level of carbon dioxide concentration and a carbon dioxide environment with a high level of carbon dioxide concentration, the low level of carbon dioxide concentration is close to a carbon dioxide concentration in a normal ventilation room, and the high level of carbon dioxide concentration is a limit value of a maximum occupational exposure in an 8-hour working day.

3. The emotional impact analysis system based on carbon dioxide exposure according to claim 1, wherein when the analysis device analyzes the electroencephalogram acquisition device, comprising following steps:
segmenting the electroencephalogram signals collected by the electroencephalogram acquisition device, corresponding to segments corresponding to tasks of each of the emotional stimulation pictures;
performing baseline correction for each of the segments, comprising subtracting an average amplitude of a baseline within a specific time window; and
processing signals after the baseline correction by Fourier transform or continuous wavelet transform, and analyzing spectrum.

4. The emotional impact analysis system based on carbon dioxide exposure according to claim 3, wherein before segmenting the electroencephalogram signals collected by the electroencephalogram acquisition device, data preprocessing is further included, comprising following steps:
removing unnecessary electrode data in the electroencephalogram signals, comprising vertical electrooculogram VEOG and horizontal electrooculogram HEOG;
adopting Butterworth filter of 0.5-40 Hz to carry out band-pass filtering on the electroencephalogram signals; and
using independent component analysis (ICA) to remove artifacts caused by eyeball movement and blinking.

5. The emotional impact analysis system based on carbon dioxide exposure according to claim 3, wherein a duration corresponding to the segments is −1 second to 2 seconds, and the specific time window is −1 second to 0 second.

6. The emotional impact analysis system based on carbon dioxide exposure according to claim 3, further comprising an outlier analysis step between the segments and the baseline correction, that is, deleting outliers with positive amplitude or negative amplitude exceeding 100.

7. The emotional impact analysis system based on carbon dioxide exposure according to claim 1, wherein for a same subject, a period of time is required to undergo experiments in different preset carbon dioxide environments.
